(19) United States
(12) Patent Application Publication
(10) Pub. No.: US 2004/0110241 A1
(43) Pub. Date: Jun. 10, 2004

(54) Materials and methods for monitoring vascular endothelial function

(76) Inventor: Mark S. Segal, Gainesville, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/313,435

(22) Filed: Dec. 6, 2002

(51) Int. Cl. G01N 33/53; G01N 33/567

(52) U.S. Cl. 435/7.21

(57) ABSTRACT

The subject invention concerns a method of monitoring a patient's vascular endothelial function by obtaining a test blood specimen from the patient; measuring the amount of circulating endothelial cells (CECs), endothelial progenitor cells (EPCs), or both within the test blood specimen; and determining the state of activation of CECs and/or EPCs within the test blood specimen. The subject invention further pertains to methods of monitoring responsiveness to a vascular therapy, such as a statin-based therapy, using the quantity and surface phenotype of CECs and/or EPCs as diagnostic or prognostic indicators of vascular health, and particularly vascular endothelial health, before, during, or after the vascular therapy.

The subject invention was made with government support under a research project supported by the National Institute of Diabetes and Digestive and Kidney Diseases of the National Institutes of Health Grant No. DK02537. The government has certain rights in this invention.

### BACKGROUND OF THE INVENTION

The endothelium is the largest organ in the body, consisting of endothelial cells lining every blood vessel. Before 1980, the endothelium had been thought of only as a vessel lining, a covering of the basement membrane, and was not thought to play role in physiological control. This changed after Furchgott and Zawadzki (Furchgott, R. F. and J. V. Zawadzki Nature, 1980, 288:373-376) demonstrated that vasodilation of a vessel in response to acetylcholine occurs only in the presence of an intact endothelium. It is now known that this vasodilation is largely the result of endothelium-derived nitric oxide. The endothelium is responsible for production of other vasodilators, such as prostacyclin (Bunting, S. et al. Prostaglandins, 1976, 12:897-913; Gryglewski, R. J. et al. Acta Biol Med Ger, 1978, 37:715-723), as well as the vasoconstrictor endothelin (Yanagisawa, M. et al. Nature, 1988, 332:411-415).

The endothelium controls tendency in a variety of ways. In the quiescent state, the endothelium exhibits antithrombotic properties by secreting prostacyclin and nitric oxide. These agents inhibit thrombosis not only vasodilation but also by the inhibition of platelet aggregation directly (Diodati, J. G. et al. Circulation, 1998, 98:17-24: Kader, K. N. et al. Tissue Eng., 2000, 6:241-251; Wu, K. K. and P. Thiagarajan Annu Rev Med., 1996, 47:315-331; Xu, W. et al. Thromb Haemost, 2001, 85:915-923). In addition, thrombomodulin expressed on endothelial cells inactivates thrombin, an activator of thrombosis. In response to low shear stress or other stimuli, endothelial cells become prothrombotic, secreting platelet-activating factor (Camussi, G. et al. J. Immunol, 1983, 131:2397-2403; McIntyre, T. M. et al. J. Clin Invest, 1985, 76:271-280; Nawroth, P. et al. Clin Haematol, 1985, 14:531-546; Prescott, S. M. et al. Proc Natl Acad Sci USA, 1984, 81:3534-3538) and expressing thromboplastin (tissue factor; factor III) on their cell surfaces (Colucci, M. et al. J. Clin Invest, 1983, 71:1893-1896; Gladal, K. S. Haemostasis, 1984, 14:378-385; Lyberg, T. et al. Br J. Haematol, 1983, 53:85-95).

There is growing evidence that the endothelium plays a crucial role in the initiation and maintenance of inflammation, standing side by side with other major protagonists of immunological response, such as T cells and monocytes. In response to a variety of stimuli, such as changes in shear stress and exposure to different environmental or hormonal changes, the expression of cell adhesion molecules on endothelial cells is increased, leading to the adherence of inflammatory cells.

Antigens expressed on the surface of endothelial cells may have an important role in immune regulation. Although human endothelial cells constitutively express major histocompatibility class (MHC) class II antigen (Rose, M. L. et al. Transplantation, 1986, 41:776-780), found on all antigen-presenting cells, the functional consequences of class II antigen expression in the absence of classic co-stimulatory molecules are still a matter of debate (Marelli-Berg, F. M. and R. I. Lechler Immunol Rev, 1999, 172:297-314).

Endothelial cells, interposed between blood and tissue, are affected by perturbations occurring in both the plasma and endothelium. In acute glomerulonephritis, the endothelium can be the target of autoantibodies, resulting in activation and apoptosis of endothelial cells. In the autoimmune disease systemic lupus erythematosus, approximately 30% of individuals have anti-endothelial cell antibodies (Song, J. et al. Rheumatol Int, 2000, 20:1-7). These antibodies can induce expression of adhesion molecules on the surface of endothelial cells, resulting in leukocyte rolling and transendothelial migration (Papa, N. D. et al. Lupus, 1999, 8:423-429, Yazici, Z. A. et al. Int Immunol, 2001, 13:349-357).

Diabetes and elevated cholesterol profoundly affect the endothelium. Prolonged hypergylycemia directly impairs endothelial cell function (Booth, G. et al. Am J. Physiol Endocrinol Metab, 2001, 280:E848-E856; Cooke, J. P. and V. J. Dzau Annu Rev. Med., 1997, 48:489-509; Garcia Soriano, F. et al. Mat Med, 2001, 7:108-113) and causes endothelial cell activation (Kado, S. et al. Life Sci, 2001, 68:727-737; Takami, S. et al. Atherosclerosis, 1998, 138:35-41; Yngen, M. et al. Blood Coagul Fibrinolysis, 2001, 12:109-116).

Elevated serum low-density protein (LDL), an independent risk factor for the development of atherosclerosis, also affects endothelial cell function. The atherosclerosis-promoting effect of LDL is likely due to its ability to increase expression of adhesion molecules on endothelial cells (Allen, S. et al. J. Clin Invest, 1998, 101:1064-1075; Allen, S. et al. FASEB J, 1998, 12:1765-1776; Lin, J. H. et al. Atherosclerosis, 1996, 127:185-194).

In thrombotic microangiopathies such as malignant hypertension, preeclampsia, radiation nephritis, and hemolytic uremic syndrome, endothelial cell activation and apoptosis may play a major pathogenic role (Mitra, D. et al. Am J. Hematol, 1998, 59:279-287; Ruggenenti, P. and G. Remuzzi J. Nephrol, 1998, 11:300-310). In each of these conditions, endothelial cell activation, arising from different stimuli, may lead to apoptosis and renal disease.

Thus, the endothelial cell participates in numerous functions of vascular physiology. Many factors, such as cytokines, can alter the surface of the endothelial cell and thereby modulate the role of the endothelium in coagulation, inflammation, vaso-regulation and adhesion. (Hebbel, R. P. et al., J. Lab. Clin. Med., 1997, 129:288; Pober, J. S., Am. J. Path., 1988, 133:426; and Favaloro, E. J., Immunol. Cell. Biol., 1993, 71:571). However, research in this area has been hindered by the inaccessibility of vascular endothelium in patients.

The presence of circulating endothelial cells (CEC) was first described in leukocyte concentrates from patients with tumors (Bouvier, C. et al. Bull Acad Suisse Sci Med, 1964, 20:15; Herbeuval, H. and M. Fourot C. R. Seances Soc Biol Fil, 1964, 158:137). In the following 25 years, a number of reports described increased numbers of CEC in response to a variety of stimuli or pathological conditions. These studies were equivocal, because endothelial cell identification was based on nonspecific, May-Grünwald Giemsa staining of leukoconcentrate after platelet depletion and red cell hemolysis (Bouvier, C. et al. Thromb Diath Haemorrh Suppl, 1970, 40:163-168; Bouvier, C. A. et al. Arch Anat Pathol, 1967, 15:133-135; Hladovec J. and P. Rossmann Thromb Res, 1973, 3:665-674; Sowemimo-Coker, S. O. et al. Am J. Hematol, 1989, 31:263-265 8, 10, 45, 100). In 1991, George et al. (George, F. et al. J. Immunol Methods, 1991, 139:65-75) unequivocally demonstrated CEC in whole blood using an endothelial cell-specific antibody. Subsequently, a number of different laboratories have identified CEC in whole blood using endothelial cell-specific monoclonal antibodies and cell culture. The numbers of CEC have been described in normal individuals and in a variety of pathological conditions.

In principle, CEC may be derived from two sources: the peripheral vasculature or the bone marrow. Cells derived from the peripheral vasculature should be more mature endothelial cells, expressing phenotypic endothelial cell markers such as von Willebrand factor, vascular endothelial cadherin (VE-cadherin), or CD146. Potential mechanisms for detachment of mature endothelial cells are mechanical disruption or apoptosis, although assays for apoptosis performed on CEC were negative (Mutin, M. et al. Blood, 1999, 93:2951-2958; Woywodt, A. et al., “Detection of circulating endothelial cells in ANCA-associated small-vessel vasculitis” In World Congress of Nephrology, San Francisco, Calif. Hagerstown, Md.: Lippincott William & Wilkins, 2001). CEC originating from the bone marrow should be derived from endothelial progenitor cells (EPC), which have differentiated to express mature endothelial cell markers. Unfortunately, although studies have addressed the origin of CEC and EPC in toto, no studies have addressed the origin of CEC specifically.

Investigators have analyzed both the numbers of CEC and EPC in a variety of clinical conditions. In normal individuals, the number of CEC is low. The number is markedly increased in conditions associated with a high degree of endothelial cell injury, such as myocardial infarction (Mutin, M. et al. Blood, 1999, 93:2951-2958), sickle cell crisis (Solovey, A. et al. N. Engl J. Med, 1997, 337:1584-1590), thrombotic thrombocytopenic purpura (Lefevre, P. et al. Thromb Haemost., Abstract, 1993, 69:522), Behcet's disease (Camoin-Jau, L. et al. Thromb Haemost, 2000, 83:631-632), diabetes (Egawhary, D. N. et al. Biochem Soc Trans, 1995, 23:402S), active systemic lupus erythematosius (Clancy, R. et al. Arthritis Rheum, 2001, 44:1203-1208; Clancy R. M. Curr Rheumatol Rep, 2000, 2:39-43), active cytomegalovirus (CMV) (Percivalle, E. et al. J. Clin Invest, 1993, 92:663-670), travascular instrumentation (Sbabati R. et al., Blood, 1991, 77:764-769; George F. et al., J. Immunol. Methods, 1991, 139:65-75), endotoxinemia (Gerrity R. G. et al., Exp. Mol. Pathol., 1976, 24:59-69) and Rickettsia conorii infection (Drancourt, M. et al. J. Infect Dis, 1992, 166:660-663; George, F. et al. Blood, 1993, 82:2109-2116), the causative agent in Mediterranean spotted fever.

In the few longitudinal studies performed, the number of CEC correlates with disease acuity. In normal donors, there are only about 2-3 CEC per ml of blood; they have a quiescent phenotype, and about 50% of them are microvascular as evidenced by CD36 positivity. Patients experiencing an acute sickle cell crisis have approximately 140 CEC/ml (Solovey, A. et al. N. Engl. J. Med., 1997, 337:1584-1590), whereas patients recovering from an acute sickle cell crisis have fewer CEC, although a number still above normal (Solovey, A. et al. N. Engl. J. Med., 1997, 337:1584-1590). Similar elevations in CEC number are seen in Mediterranean spotted fever (George, F. et al. Blood, 1993, 82:2109-2116) and CMV infection (Percivalle, E. et al. J. Clin Invest, 1993, 92:663-670). In immunocompromised patients with disseminated CMV infection, a CEC number greater than 10/ml is associated with higher viral loads and a higher level of antigenemia (Percivalle, E. et al. J. Clin Invest, 1993, 92:663-670).

Although an increased CEC number is associated with conditions that either activate or injure the endothelium, it is unlikely that release of endothelial cells from the peripheral vasculature accounts for all of this increase. In a 154-lb. human with a blood volume of 4.7 liters, an increase in CEC by approximately 15 cells/ml, as occurs in acute myocardial infarction (Mutin, M. et al. Blood, 1999, 93:2951-2958), yields a total of 70,500 additional CEC. A minimum of 3.2 cm of vessel surface would be required to release this number of cells. This area is far larger than expected for the endothelial injury occurring in a myocardial infarction. More likely, the injury to the endothelial cells lining the coronary vessel results in the release of endothelial cells into the circulation as well as the release of a signal accelerating the maturation of EPC to mature endothelial cells.

The signal(s) involved in increasing the number of CEC derived from the bone marrow has yet to be identified, although likely candidates include transforming growth factor-α, basic fibroblastic growth factor, and VEGF. Transfection of ischemic myocardium with a plasmid producing a 3.5-fold increase in the amount of circulating VEGF increases the number of EPC 3.5-fold (Kalka, C. et al. Ann Thorac Surg, 2000, 70:829-834). VEGF levels in burn and coronary artery-bypass patients correlate with EPC number (Gill, M. et al. Circ Res, 2001, 88:167-174). Medications also affect EPC number. In mice, 3-hydroxy-3-methylglutaryl-CoA reductase inhibitors increase the number of EPC by activating the phosphatidylinositol-3 kinase/AKT pathway (Dimmeler, S. et al. J. Clin Invest, 2001, 108:391-397; Llevadot, J. et al. J. Clin Invest, 2001, 108:399-405).

Although CEC have not yet been directly utilized in any clinical studies, CEC potentially have a number of diagnostic and therapeutic uses. CEC number may reflect disease activity in several renal diseases, for example.

CEC properties may provide a means of monitoring the functional state of the endothelium. CEC may be exposed to some of the same environmental stimuli and activating factors as the vessel wall endothelium, and they may be similarly affected. Stimulated endothelial cells increase the expression of surface adhesion molecules, express and release procoagulant factors, and secrete vasoactive compounds including endothelins, nitric oxide, and prostaglandins. All of these substances have been implicated in the pathophysiology of a variety of diseases (Corti, R. et al. “Endothelial dysfunction and hypertension” In: Vascular Endothelium in Human Physiology and Pathophysiology, edited by Vallance P. J. T. and Webb D. J. Amsterdam: Harwood, 2000, pp. 109-128; Goligorsky, M. S. et al. Hypertension, 2001, 37:744-874; Haller, H. Drugs, 1997, 53:1-10; Naicker, S. and K. D. Bhoola Pharmacol Ther, 2001, 90:61-88; Smith, C. W. Can J. Physiol Pharmacol, 1993 71:76-87; Vapaatalo, H. and E. Mervaala Med Sci Monit, 2001, 7:1075-1085). Measurements of the state of activation of CEC may provide insights into the health of the vascular endothelium. In sickle cell crises, CEC tissue factor expression is seven times that observed in normal controls (Solovey, A. et al. J. Clin Invest, 1998, 101:1899-1904). CEC may provide an index of oxidative stress. Heme oxygenase-1, an enzyme increased in response to oxidative stress, is markedly increased in the CEC of sickle cell patients compared with healthy controls (Nath, K. A. et al. Am J. Pathol, 2001, 158:893-903).

Having the ability to monitor the state of the vascular endothelium may provide insight into the pathophysiology of the array of diseases thought to involve endothelial cell dysfunction. The state of the vascular endothelium may be also used to evaluate the effectiveness of drug therapy targeted to diseases of the vascular endothelium.

### BRIEF SUMMARY OF THE INVENTION

The subject invention pertains to the utilization of circulating endothelial cells (CECs) and/or endothelial progenitor cells (EPCs) as ex vivo indicators of vascular health. Because CECs and EPCs are subjected to the same environmental stimuli and blood-born activating factors as vessel wall endothelium, they can provide a direct and non-invasive method to analyze the phenotype and function of the vascular endothelium.

In one aspect, the subject invention pertains to a method of monitoring a patient's vascular endothelial function by obtaining a test blood specimen from the patient; measuring the amount of CECs within the test blood specimen; and determining the state of activation of endothelial cells (CECs and/or EPCs) within the test blood specimen. According to the methods of the subject invention, the state of activation of the CECs and/or EPCs can be determined by measuring one or more endothelial cell activation markers within the test blood specimen, assigning the amount of endothelial cell activation marker(s) a value(s), and normalizing the assigned value(s) of the endothelial cell activation marker(s) by dividing the value(s) by the number of CECs and/or EPCs determined to be within the test blood specimen. Cell markers that are characteristic of an “activated” endothelial cell phenotype include, but are not limited to, surface adhesion molecules, procoagulant factors, endothelins, growth factor receptors, nitric oxide, endothelial nitric oxide synthase (eNOS), inducible nitric oxide synthase (iNOS), prostaglandins 12 (PGI2), tissue factors, heme oxygenase, tissue plasminogen activator (tPA), mitochondria superoxide dismutase (MnSOD), Cu/Zn superoxide dismutase (Cu/Zn SOD), tumor growth factor-beta (TGF-β), cyclooxygenase 1 (COX-1), cyclooxygenase-2 (COX-2), vascular cell adhesion molecule (VCAM), such as VCAM-1, intercellular adhesion molecule (ICAM), such as ICAM-1, vascular endothelial growth factor (VEGF), VEGF receptor, E-selectin, and P-selectin. These markers can be produced by CECs and/or EPCs. In one embodiment, VEGF receptors expressed by EPCs are measured.

The subject invention further pertains to methods of monitoring responsiveness to a vascular therapy, such as a statin-based therapy, using the quantity and surface phenotype of endothelial cells (CECs and/or EPCs) as diagnostic or prognostic indicators of vascular health, and particularly vascular endothelial health, before, during, or after the vascular therapy. In one embodiment, the method involves administering a therapeutic vascular agent, such as a statin, to the patient; obtaining a test blood specimen from the patient; measuring the amount of the therapeutic vascular agent within the blood specimen; measuring the amount of RNA encoding an endothelial cell activation marker within the test blood specimen; measuring the amount of endothelial cell activation marker enzymatic activity, if the endothelial cell activation marker is an enzyme; measuring the amount of endothelial cells (CECs and/or EPCs) within the test blood specimen; correlating at least one of the following with the amount of endothelial cells and therapeutic vascular agent within the test blood specimen: (i) amount of RNA encoding an endothelial cell activation marker; and (ii) amount of endothelial cell activation marker enzymatic activity; and, optionally, maintaining or modulating the dosage of subsequent administrations of the therapeutic vascular agent to the patient. The dosage of the therapeutic vascular agent can be decreased, such as down to zero, increased, or maintained. A different therapeutic vascular agent, such as a statin or non-statin drug, can be administered, either to replace or supplement the therapeutic vascular agent currently administered to the patient.

An additional aspect of the instant invention involves a kit that includes at least two of the following components: a means for measuring the amount of a therapeutic vascular agent within a test blood specimen; a means for measuring the amount of RNA encoding an endothelial cell activation marker within the test blood specimen; a means for measuring the amount of endothelial cell activation marker enzymatic activity within the test blood specimen; and a means for measuring the amount of endothelial cells (CECs and/or EPCs) within the test blood specimen.

### DETAILED DISCLOSURE OF THE INVENTION

The subject invention pertains to a method of monitoring a patient's vascular endothelial function by accessing a patient's peripheral circulation in a non-invasive fashion in order to determine the status of endothelial cells within the patient. Advantageously, using the methods of the subject invention, the course of a particular vascular disease can be predicted, appropriate therapeutic strategies can be developed and their effectiveness more accurately accessed.

In one aspect, the subject invention pertains to a method of monitoring a patient's vascular endothelial function by obtaining a test blood specimen from the patient; measuring the amount of endothelial cells (circulating endothelial cells (CECs) and/or endothelial progenitor cells (EPCs)) within the test blood specimen; and determining the state of activation of endothelial cells within the test blood specimen. According to the methods of the subject invention, the state of activation of the endothelial cells can be determined by measuring one or more endothelial cell activation markers within the test blood specimen, assigning the amount of endothelial activation marker(s) a value(s), and normalizing the assigned value(s) of the endothelial activation marker(s) by dividing the value(s) by the number of endothelial cells determined to be within the test blood specimen. Thus, a value is generated representing the quantity of endothelial cell activation marker(s) present per unit of endothelial cells (i.e., per unit of CECs and/or EPCs).

Meaningful ratios of specific activation marker(s) and numbers of endothelial cells can be differentiated by studying individuals at risk for cardiovascular events. The number of endothelial cells and amount and/or activity of one or more activation markers can be determined at a given time point. The amount and/or activity of a given activation marker or markers per unit of endothelial cells (index of activation) can then be correlated with future events, providing indices of risk for a future cardiovascular event. An index of activation can also be determined for patients that are not currently taking medications (e.g., therapeutic vascular agents) and subsequently determined following administration of one or more therapeutic vascular agents.

As used herein, the term “endothelial cell activation marker” refers to an expressed gene product (e.g., a protein) or activation of an enzymatic activity that is associated with a change in the state of the endothelium that is vasodilatory and/or antithrombotic to one that is vasoconstrictive and/or prothrombotic (Malek, A. M. et al., JAMA, 1999, 282(21):2035-2042). Cell markers that are characteristic of an “activated” endothelial cell phenotype include, but are not limited to, surface adhesion molecules, procoagulant factors, endothelins, growth factor receptors, nitric oxide, endothelial nitric oxide synthase (eNOS), inducible nitric oxide synthase (iNOS), prostaglandins 12 (PGI2), tissue factors, heme oxygenase (HO), such as HO-1, tissue plasminogen activator (tPA), mitochondria superoxide dismutase (MnSOD), Cu/Zn superoxide dismutase (Cu/Zn SOD), tumor growth factor-beta (TGF-β), cyclooxygenase 1 (COX-1), cyclooxygenase-2 (COX-2), vascular cell adhesion molecule (VCAM), such as VCAM-1, intercellular adhesion molecule (ICAM), such as ICAM-1, vascular endothelial growth factor (VEGF), VEGF receptor, E-selectin, and P-selectin. For example, the activated endothelial state can include one or more of the following: production of endothelin, decrease in nitric oxide (NO), decrease in tissue plasminogen activator, thrombomodulin, PGI2, release of von Willebrand factor, increase in VCAM-1, E-selectin, and plasminogen activator iihibitor-1.

As indicated above, endothelial cell activation or endothelial cell activity can be determined by measuring one or more endothelial cell activation markers within the test blood specimen. This can be accomplished in a number of different ways. For exainple, an increase in the amount of a endothelial cell activation marker protein or an increase in the activity of the protein (while maintaining a constant level of the protein) can result in increased “activity”. An increase in the amount of protein can result from increased transcription of the endothelial cell activation marker gene, increased stability of the endothelial activation marker mRNA, or a decrease in endothelial cell activation marker protein degradation.

A direct measure of an endothelial cell activation marker would be to measure the amount of endothelial cell activation marker present. Where the endothelial cell activation marker is an enzyme, such as nitric oxide synthase, another direct measure would be to measure the conversion of enzymatic substrate to enzymatic product or bi-product. The endothelial cell activation marker activity can also be measured more indirectly, for example, by measuring mRNA half-life (an upstream indicator) or by a phenotypic response to the presence of the enzymatic substrate (a downstream indicator), product, or bi-product.

For example, “eNOS activity” is intended to mean the ability of a cell to generate nitric oxide from the substrate L-arginine. Increased eNOS activity can be accomplished in a number of different ways. For example, an increase in the amount of eNOS protein or an increase in the activity of the protein (while maintaining a constant level of the protein) can result in increased “activity”. An increase in the amount of protein available can result from increased transcription of the eNOS gene, increased stability of the eNOS mRNA or a decrease in eNOS protein degradation.

Normal baseline levels are the amounts (inclusive of zero) of a given endothelial cell activation marker in a normal control group, preferably controlled for age and having no symptoms which would indicate alteration of vascular endothelial function. The actual level then will depend upon the particular age group selected and the particular measure employed to assay the endothelial cell activation marker.

Expression of endothelial cell activation markers, such as circulating endothelial cell surface molecules, in response to a vascular disease condition correlate with the presence of that condition and with the success of the particular treatment regimen prescribed. For example, in the case of atherosclerosis, antibody or polynucleotide probes can be applied to a blood specimen to quantify the expression of circulating endothelial cell activation markers and thus predict the course of disease and/or determine appropriate therapeutic strategies, or assess their effectiveness.

In one embodiment, the method involves comparing an individual's endothelial cell activation marker pattern with endothelial cell activation marker patterns of individuals who have been shown to respond positively or negatively to a particular therapeutic regimen. Endothelial cell activation marker patterns can vary with time, treatment, age, presence of disease risk factors, etc. The term “therapeutic regimen”, as used herein, refers to treatments aimed at the elimination or amelioration of symptoms and events associated with vascular disease conditions. In addition to pharmaceutical interventions, such treatments can include without limitation one or more of alteration in diet, lifestyle, and exercise regimen; invasive and noninvasive surgical techniques such as atherectomy, angioplasty, and coronary bypass surgery. Pharmaceutical interventions can include administration of a therapeutic vascular agent such as, for example, angiotensin converting enzyme (ACE) inhibitors, angiotensin II receptor antagonists, diuretics, alpha-adrenoreceptor antagonists, cardiac glycosides, phosphodiesterase inhibitors, beta-adrenoreceptor antagonists, calcium channel blockers, 3-hydroxy-3-methylglutaryl-coenzyme A (HMG-CoA) reductase inhibitors, imidazoline receptor blockers, endothelin receptor blockers, and organic nitrites. Interventions with pharmaceutical agents not yet known whose activity correlates with particular circulating endothelial cell activation marker patterns associated with vascular disease are also encompassed.

The present invention exploits the observation that particular endothelial cell marker patterns correlate with an individual's responsivity to therapeutic vascular agents, such as ACE inhibitors and HMG CoA reductase inhibitors. It is contemplated, for example, that patients who are candidates for a particular therapeutic regimen will be screened for endothelial cell activation marker patterns that correlate with responsivity to that particular therapeutic regimen.

Blood specimens can be obtained using methods of sampling known in the art, such as venipuncture. Preferably, the initial blood obtained from venipuncture is not used. Venous blood can be drawn into vessels, such as tubes, and optionally treated with silicon and EDTA for subsequent analysis. As used herein, the terms “blood sample” and “blood specimen” are used interchangeably to refer to a volume of blood that is preferably, although not necessarily, removed from the patient's peripheral circulatory system.

Preferably, the blood specimen is obtained by isolating a volume of blood from the bloodstream. However, other means for sampling the patient's blood can be utilized, such as in-dwelling systems. For example, an in-dwelling catheter can be utilized to remove a volume of blood. Alternatively, blood can be sampled and the endothelial cell activation marker(s) and therapeutic vascular agent(s) can be measured in vivo using a so-called “lab on a chip” apparatus.

In one embodiment of the method of the subject invention, the blood specimen is treated to remove lower molecular weight contaminants, for example, by dialysis. The term “dialysis” is intended to include any technique of separating the endothelial cell activation markers and, optionally, a therapeutic vascular agent, in the sample from lower molecular weight contaminants. Spectra/Por membrane dialysis tubing with a desired molecular weight cut-off (MWCO) can be utilized. Other products that can be utilized include hollow fiber concentration systems consisting of regenerated cellulose fibers for larger volumes; a multiple dialyzer apparatus with a sample size for one to five milliliters; and a multiple microdializer apparatus, convenient for samples in plates with 96 wells, for example. Other equivalent techniques include passage through a column holding a resin or mixture of resins suitable for removal of low molecular weight materials. Resins such as BIOGEL (BIORAD, Hercules, Calif.) and SEPHAROSE (PHARMACIA, Piscataway, N.J.) and others are well-known to the skilled artisan. The technique of dialysis, or equivalent techniques with the same function, are intended to remove low molecular weight contaminants from the blood specimen. Although not an essential component of the subject invention, the step of removal of such contaminants may facilitate detection of the CEC activation markers and therapeutic vascular agents within the blood sample.

Specific antibodies (e.g., polyclonal or monoclonal antibodies) raised to an endothelial cell activation marker can be used to detect that component in blood specimens. The term “antibody” as used herein, includes monoclonal and polyclonal antibodies as well as antibody fragments which bind specifically but reversibly to the described epitope. It is preferred that the antibody or antibody fragment is derived from a monoclonal antibody or antibody fragment. Preparation of monoclonal and polyclonal antibodies to an antigen representing an endothelial cell activation marker can be achieved using any known method, and for example, those described in Zola, H. (1988) “Monoclonal Antibodies—A manual of techniques” CRC Press, and Antibodies: A Laboratory Manual, Harlow & Lane; Cold Spring Harbor (1988), incorporated herein by reference. Specific high affinity binding proteins can be used in place of antibodies, and can be made according to methods known to those in the art. For example, proteins that bind specific DNA sequences may be engineered (U.S. Pat. No. 5,096,815), and proteins that bind a variety of other targets, particularly protein targets (U.S. Pat. No. 5,233,409; U.S. Pat. No. 5,403,484) can be engineered and used in the present invention. Antibodies and/or binding proteins can be incorporated into large scale diagnostic or assay protocols that can involve immobilization of the antibody or binding protein onto a surface, such as a multi-well plate assay, or on beads, for example.

General techniques to be used in performing various immunoassays are known to those of ordinary skill in the art. General descriptions of these procedures are provided in manuals of the art (Ishikawa, E. et al., (1988) Enzyme Immunoassay, Igaku-shoin, Tokyo, N.Y.; Hallow, E. et al., Antibodies: A Laboratory Manual, CSH Press, N.Y.).

According to the methods of the subject invention, RNA can be isolated from endothelial cells (CECs and/or EPCs) within a blood specimen using standard methods well known to those of ordinary skill in the art such as guanidium thiocyanate-phenol-chloroform extraction (Chomocyznski et al., Anal Biochem., 1987, 162:156.). The isolated RNA is then subjected to coupled reverse transcription and amplification by polymerase chain reaction (RT-PCR), using specific oligonucleotide primers. Conditions for primer annealing are chosen to ensure specific reverse transcription and amplification; thus, the appearance of an amplification product is diagnostic of the presence of particular endothelial cell activation markers. In another embodiment, RNA is reverse-transcribed and amplified, after which the amplified sequences are identified by, e.g., direct sequencing.

RNA based nucleic acid amplification assays, including the reverse transcriptase polymerase chain reaction (RT-PCR, also known as reverse transcription polymerase chain reaction or RNA-PCR), branched DNA signal amplification, and self-sustained sequence replication assays, such as isothermal nucleic acid sequence based amplification (NASBA), have proven to be highly sensitive and specific methods for detecting small numbers of RNA molecules. As such, they can be used in direct assays of blood. Since peripheral blood is readily obtainable from patients with cardiovascular disease, for example, and circulating endothelial cells are known to circulate in the blood of patients with cardiovascular disease, R1-PCR can be utilized to detect intracellular RNA extracted from circulating endothelial cells and/or endothelial progenitor cells.

An additional aspect of the instant invention involves a kit that includes at least two of the following components: a means for measuring the amount of a therapeutic vascular agent within a test blood specimen; a means for measuring the amount of RNA encoding an endothelial cell activation marker within the test blood specimen; a means for measuring the amount of endothelial cell activation marker enzymatic activity within the test blood specimen; and a means for measuring the amount of endothelial cells (CECs and/or EPCs) within the test blood specimen. The components of the kit can include any of those materials described herein for measurement of therapeutic vascular agents, RNA encoding an endothelial cell activation marker, the amount of endothelial cell activation marker enzymatic activity, and the amount of endothelial cells, or other materials capable of measuring these cells, nucleic acids, proteins, and/or agents. Optionally, each of the kit's components can be contained within a solution. Optionally, the kit's components can be compartmentalized within packaging. For example, the kit can include one or more solid supports to which is fixed one or more antibodies specific for a therapeutic vascular agent, an endothelial cell surface antigen, and/or an endothelial cell activation marker. Furthermore, the kit can include a means for determining the enzymatic activity of an endothelial cell activity marker, such as a diaminofluorescein (DAF) compound.

Therapeutic Vascular Agents

The conversion of 3-hydroxy-3-methylglutaryl-coenzyme A (HMG-CoA) to mevalonate is an early rate-limiting step in the cholesterol biosynthetic pathway. This step is catalyzed by the enzyme HMG-CoA reductase. Statins inhibit HMG-CoA reductase from catalyzing this conversion and, therefore, inhibit the synthesis of cholesterol. As such, statins are collectively potent lipid lowering agents. There are many compounds described in the art that have been obtained naturally or synthetically, which have been seen to inhibit HMG-CoA reductase, and which for this category of therapeutic vascular agents useful for practicing the subject invention. Examples of HMG-CoA reductase inhibitors that may be used include but are not limited to lovastatin (see U.S. Pat. No. 4,231,938), simvastatin (see U.S. Pat. No. 4,444,784), pravastatin (see U.S. Pat. No. 4,346,227), fluvastatin (see U.S. Pat. No. 5,354,772), atorvastatin (see U.S. Pat. No. 5,273,995), and cerivastatin (also known as rivastatin; see U.S. Pat. No. 5,177,080). The structural formulas of these and additional HMG-CoA reductase inhibitors that may be used in the methods of the subject invention are known in the art.

The term “HMG-CoA reductase inhibitor” is intended to include all pharmaceutically acceptable salt, ester, and lactone forms of compounds which have HMG-CoA reductase inhibitory activity, and therefore the use of such salts, esters, and lactone forms is included within the methods of the subject invention. Compounds which have inhibitory activity for HMG-CoA reductase can be readily identified by using assays well-known in the art, such as those described in U.S. Pat. No. 4,231,938. While the HMG-CoA reductase inhibitor can be administered orally or parenterally, for example, oral dosing is preferred.

Therapeutically effective amounts of the therapeutic vascular agents are suitable for use in the methods of the present invention. The term “therapeutically effective amount” is intended to mean that amount of a drug or pharmaceutical agent that will elicit the biological or medical response of a tissue, a system, animal, or human, that is being sought by a researcher, veterinarian, medical doctor or other clinician. The initial dosage regimen utilizing a therapeutic vascular agent, such as an HMG-CoA reductase inhibitor, is selected in accordance with a variety of factors including type, species, age, weight, sex, and medical condition of the patient; the severity of the condition to be treated; the rout of administration; the renal and hepatic function of the patient; and the particular compound or salt or ester thereof employed. If two or more different active agents are being used together in a combination therapy, the potency of each of the agents and the enhanced effects achieved by combining them together must also be taken into account. A consideration of these factors is well within the purview of the ordinarily skilled clinician for the purpose of determining the therapeutically effective amounts of the drug combination needed to prevent, counter, or arrest the progress of the condition.

Dosage information for known therapeutic vascular agents, such as HMG-CoA reductase inhibitors, is well known in the art. For example, several HMG-CoA reductase inhibitors are marketed in the United States. In particular, the daily dosage amounts of the HMG-CoA reductase inhibitor may be the same or similar to those amounts which are employed for anti-hypercholesterolemic treatment and which are described in the Physicians' Desk Reference (PDR). For example, see the 50^{th} Ed. of the PDR, 1996 (Medical Economics Co.); in particular, see at page 216 the heading “Hypolipidemics,” sub-heading “HMG-CoA Reductase Inhibitors,” and the reference pages cited therein. Preferably, the initial oral dosage amount of HMG-CoA reductase inhibitor is from about 1 to 200 mg/day, and more preferably from about 5 to 160 mg/day. However, dosage amounts will vary depending on the potency of the specific HMG-CoA reductase inhibitor used as well as other factors noted above. An HMG-Coa reductase inhibitor that has sufficiently greater potency may be given in sub-milligram daily dosages.

Angiotensin converting enzyme (ACE) inhibitors are compounds that prevent the conversion of angiotensin I into the pressor-active angiotensin II. ACE inhibitors include amino acids and derivatives thereof, peptides, including di and tri peptides and antibodies to ACE which intervene in the rennin-angiotensin system by inhibiting the activity of ACE thereby reducing or eliminating the formation of pressor substance angiotensis II. ACE inhibitors have been used medically to treat hypertension, congestive heart failure, myocardial infarction, and renal disease. Classes of compounds known to be useful as ACE inhibitors include acylmercapto and mercaptoalkanoyl prolines, such as captopril (U.S. Pat. No. 4,105,776) and zofenopril (U.S. Pat. No. 4,316,906) carboxyalkyl dipeptides, such as enalapril (U.S. Pat. No. 4,374,829), lisinopril (U.S. Pat. No. 4,374,829), quinapril (U.S. Pat. No. 4,344,949), ramapril (U.S. Pat. No. 4,587,258), and perindopril (U.S. Pat. No. 4,508,729), carboxyalkyl dipeptide mimics, such as cilazapril (U.S. Pat. No. 4,512,924) and benazapril (U.S. Pat. No. 4,410,520), phosphinylalkanoyl prolines, such as fosinopril (U.S. Pat. No. 4,337,201) and trandolopril. Other compounds are, for example, described in the following patents: U.S. Pat. Nos. 4,350,633; 4,294,832; and 4,350,704. Further examples of ACE inhibitors include benazeprilat, delapril, fentiapril, imidapril, libenzapril, moexipril, pentopril, perindopril, pivopril, quinaprilat, spirapril, spiraprilat, ceronapril, enalapril, indolapril, and alacepril. Such ACE inhibitors or a pharmaceutically acceptable salt thereof are appropriate for use in the subject invention.

The doses of an ACE inhibitor which can be administered in accordance with the present invention depends on several factors such as, for example, the age, weight, and the severity of the condition under treatment, as well as the route of administration, dosage form and regimen and the desired result, and additionally the potency of the particular ACE inhibitor employed in the composition. In addition, account should be taken of the recommended maximum daily dosages for the ACE inhibitors. A unit dosage formulation such as a tablet or capsule will usually contain, for example, from 0.1 mg to 500 mg of an ACE inhibitor. Preferably, a unit dose formulation will contain 0.1 to 100 mg of an ACE inhibitor.

According to the method of the subject invention, the pharmaceutical vascular agents may be administered up to six times daily, conveniently 1 to 4 times daily and preferably 1 to 2 times daily, so that a dose of the ACE inhibitor in the general range of 0.01 to 100 mg/kg, preferably 0.1 to 10 mg/kg, more preferably 0.1 to 5 mg/kg, is administered daily and a dose of ACE inhibitor in the general range 0.01 to 100 mg/kg, preferably 0.01 to 20 mg/kg, more preferably 0.01 to 1 mg/kg, is administered daily.

Table 1 shows a number of potentially therapeutic vascular agents that may improve, in part or in whole, vascular endothelial function. Table 1 is not exhaustive, and is only illustrative of the many medical treatments that can be applied to improve vascular endothelial performance.

TABLE 1

| | |
|---|---|
| Anticoagulants (such as | e.g., dicumarol, cumarine derivatives, |
| those for treatment of | heparin calcium, heparin sodium, and |
| deep venous thrombosis or | warfarin sodium |
| pulmonary embolism) | |
| Antihypertensives | |
| Alpha-adrenergic blocks | e.g., bunazosin, phenoxybenzamine |
| | hydrochloride, phentolamine mesylate, |
| | prazosin hydrochloride, terazosin |
| | hydrochloride, tolazoline hydrochloride, |
| | and urapidil |
| Angiotensin-converting | e.g., benazapril, captopril, cilazapril |
| enzyme inhibitors | enalaprilat, enalapril maleat, fornopril, |
| | fosinopril, lisinopril, nonopril, perindropril, |
| | quinapril, ramapril, trandolopril, and |
| | zofenopril |
| Beta-adrenergic blockers | (see under “cardiovascular agents”) |
| Calcium channel blockers | (see under “cardiovascular agents”) |
| Centrally acting anti- | e.g., alphamethyldopa, clonidine, |
| hypertensives | guanfacine, rilmenidine, and guanobenz |
| 3-hydroxy-3methylglutaryl- | e.g., lovastatin, simvastatin, pravastatin, |
| coenxyme A | fluvastatin, atorvastatin, and cerivastatin |
| (HMG-CaA) inhibitors | |
| Monoamine oxidase | e.g., pargyline hydrochloride |
| inhibitors | |
| Miscellaneous | e.g., clonidine hydrochloride, diazoxide, |
| | guanabenz acetate, guanadrel, sulfate, |
| | guanethidine sulfate, guanfacine |
| | hydrochloride, hydralazine hydrochloride, |
| | mecamylamine, hydrochloride, |
| | methyldopa, metyrosine, minoxidil, |
| | nitroprusside sodium, and trimethaphan |
| | camsylate |
| Rauwolfia alkaloids | e.g., deserpidine, rauwolfa serpentina, |
| | rescinnamine, and reserpine |
| Cardiovascular agents | |
| (see also listing for | |
| antihypertensives) | |
| Antiarrhythmics and | e.g., adenosine, amiodarone hydrochloride, |
| miscellaneous | bretylium tosylate, disopyramide, |
| | phosphate, encainide, hydrochloride, |
| | flecainide acetate, indecainide |
| | hydrochloride, lidocaine, lidocaine |
| | hydrochloride, mexiletine, hydrochloride, |
| | molsidomine, procainamide hydrochloride, |
| | propafenone, hydrochloride, propanolol, |
| | guinidine gluconate, guinidine, |
| | polyglacturonate, quidinidine sulfate, |
| | sotalol, and tocainide |
| Anticholinergics | e.g., atropine sulfate |
| Beta-adrenergic blockers | e.g., acebutolol, atenolol, betaxolol, |
| | bisoprolol, labetalol, metoprolol, tartrate, |
| | nadolol, oxprenolol, pindolol, propanolol |
| | hydrochloride, sotalol, and timolol maleate |
| Calcium channel blockers | e.g., amlodipine, diltiazem hydrochloride, |
| | felodipine, isladipine, lacadipine, |
| | nicardipine, nifedipine, nitrendipine, and |
| | verapamil hydrochloride |
| Cardiac glycosides | e.g., deslanoside, digitalis glycoside, |
| | digitoxin, digoxin, and strophantin |
| Hydantoin derivatives | e.g., phenytoin sodium |
| Nitrates | e.g., nitroglycerin, isosorbide, |
| | pentaerythritol tetranitrate, and erythrityl |
| | tetranitrate |
| Phosphodiesterase | e.g., methylxanthines |
| inhibitors | |
| Thrombolytics | e.g., streptokinase, urokinase, tissue |
| | plasminogen activator (tPA), and |
| | anisoylated plasminogen, streptokinase |
| | activator complex (APSAC) |
| Vasodilators and vaso- | |
| constrictors (see under | |
| “Antihypertensives” | |
| and “Vasoactive | |
| Substances”) | |
| Diuretics | |
| Aldosteron antagonists | e.g., amiloride, canrenone, spironolactone, |
| and potassium | and triamterene |
| sparing diuretics | |
| Carbonic anhydrase | e.g., acetazolamide, acetazolamide sodium, |
| inhibitors | dichlorphenamide, and methazolamide |
| Loop diuretics | e.g., bumetanide, ethacrynate sodium, |
| | ethacrynic acid, furosemide, and torsemide |
| Miscellaneous | e.g., alcohol and caffeine |
| Natural medicinal products | e.g., terminalia arjuna and moringo oleifera |
| Osmotic agents | e.g., mannitol, glycerin and hyperosmolar |
| | solution |
| Plasma expanders | e.g., dextran |
| Thiazides | e.g., bendroflumethiazide, benzthiazide, |
| | chlorothiazide, cyclothiazide, |
| | hydrochlorothiazide, hydroflumethiazide, |
| | indapamide, methyclothiazide, |
| | polythiazide, and trichlormethiazide |
| Thiazide-like agents | e.g., chlorthalidone, metolazone, and |
| | quinethazone |
| Serum albumin | |
| Vasoactive substances | e.g., bamethan, bencyclane, bethahistine, |
| | cyclandelate, cinnarizine, citicoline, |
| | dihydroergocristine, dihydroergotoxine, |
| | dipyridamole, ebunamonine, flunarizine, |
| | ginko-biloba extracts, horse-chestnut seed |
| | extract, isoxsuprine, naftidrofuryl, |
| | nicergoline, nicotinic aid derivatives, |
| | nylidrin, oxerutins, i.e., hydroxethyl |
| | derivatives of rutin, pentoxifylline, |
| | papaverine, piracetam, piribedil, raubasine, |
| | suloctidil, and vincamine |

Pharmaceutically acceptable salts of the therapeutic vascular agents, in accordance with the present invention, are the salts with physiologically acceptable bases and/or acids well known to those skilled in the art of pharmaceutical technique. Suitable salts with physiologically acceptable bases include, for example, alkali metal and alkaline earth metal salts, such as sodium, potassium, calcium and magnesium salts, and ammonium salts and salts with suitable organic bases, such as methylamine, dimethylamine, trimethylamine, piperidine, morpholine and triethanolamine. Suitable salts with physiologically acceptable acids include, for example, salts with inorganic acids such as hydrohalides (especially hydrochlorides or hydrobromides), sulphates and phosphates, and salts with organic acids.

Monitoring of vascular endothelial function is also particularly relevant in many critical care situations including patients with acquired immunodeficiency syndrome (AIDS), autoimmune disorders, bums, bacteremia, cancer leading to local or distant organ failure, cardiac arrest, coma, drowning or near-drowning, drug-induced complications, drug overdose, heart failure, hepatic failure, infections, inhalation of toxic substances, intestinal ischemia and infarction, myocardial ischemia or infarction, poisoning, prolonged non-ambulatory convalescence, pulmonary embolism, renal failure, respiratory arrest, trauma, transplant complications, sepsis, shock, and arterial or venous thrombosis. The use of continuous monitoring, as described in further detail herein, will be particularly applicable in this clinical setting.

In another aspect, the invention is directed to a method of treating cardiovascular and related diseases, for example, hypertension, hypertrophy, arrhythmia, congestive heart failure, myocardial ischemia, heart failure subsequent to myocardial infarction, myocardial infarction, ischemia reperfusion injury, blood coagulation, and platelet aggregation. For such a method, a therapeutic vascular agent, such as a statin or ACE inhibitor can be administered alone or concurrently with another therapeutic vascular agent, such as another statin or ACE inhibitor or, for example, an angiotensin II receptor antagonist, a vasodilator, a diuretic, an alpha-adrenergic receptor antagonist, an antioxidant, or a mixture thereof.

The term “heart disease” is used herein in the general sense and includes conditions ranging, for example, from those in which positive inotropic medications are useful to those in which coronary vessel occlusion is predominant, to arrhythmias or cardiotoxicity, such as that which may be observed as a side effect of cardiotoxic drugs, e.g., doxorubicin.

As used herein, the term “heart failure” is intended to describe a pathophysiological condition in which the heart is unable to pump blood at a rate commensurate with the requirement of the metabolizing tissues or can do so only from an elevated filling pressure (increased load). Thus, the heart has a diminished ability to keep up with its workload. Over time, this condition can lead to excess fluid accumulation, such as peripheral edema, and is referred to as congestive heart failure.

When an excessive pressure or volume load is imposed on a ventricle, myocardial hypertrophy (i.e., enlargement of the heart muscle) develops as a compensatory mechanism. Hypertrophy permits the ventricle to sustain an increased load because the heart muscle can contract with greater force. However, a ventricle subjected to an abnormally elevated load for a prolonged period eventually fails to sustain an increased load despite the presence of ventricular hypertrophy, and pump failure can ultimately occur.

Heart failure can arise from any disease that affects the heart and interferes with circulation. For example, a disease that increases the heart muscle's workload, such as hypertension, will eventually weaken the force of the heart's contraction. Hypertension is a condition in which there is an increase in resistance to blood flow through the vascular system. This resistance leads to increases in systolic and/or diastolic blood pressures. Hypertension places increased tension on the left ventricular myocardium, causing it to stiffen and hypertrophy, and accelerates the development of atherosclerosis in the coronary arteries. The combination of increased demand and lessened supply increases the likelihood of myocardial ischemia leading to myocardial infarction, sudden death, arrhythmias, and congestive heart failure.

As used herein, the term “ischemia” refers to a condition in which an organ or a part of the body fails to receive a sufficient blood supply. When an organ is deprived of a blood supply, it is said to be hypoxic. An organ will become hypoxic even when the blood supply temporarily ceases, such as during a surgical procedure or during temporary artery blockage. Ischemia initially leads to a decrease in or loss of contractile activity. When the organ affected is the heart, this condition is known as myocardial ischemia, and myocardial ischemia initially leads to abnormal electrical activity. This can generate an arrhythmia. When myocardial ischemia is of sufficient severity and duration, cell injury can progress to cell death, i.e., myocardial infarction—and subsequently to heart failure, hypertrophy, or congestive heart failure.

When blood flow resumes to an organ after temporary cessation, this is known as ischemic reperfusion of the organ. For example, reperfusion of an ischemic myocardium can counter the effects of coronary occlusion, a condition that leads to myocardial ischemia. Ischemic reperfusion to the myocardium can lead to reperfusion arrhythmia or reperfusion injury. The severity of reperfusion injury is affected by numerous factors, such as, for example, duration of ischemia, severity of ischemia, and speed of reperfusion. Conditions observed with ischemia reperfusion injury include neutrophil infiltration, necrosis, and apoptosis.

Disorders associated with vasoconstriction or other biological effects of endothelin and/or angiotensin II which may be particularly mentioned are the control or prevention of coronary disorders, cardiovascular disorders such as hypertension, heart failure, ischemia (in the heart, brain, gastrointestinal tract, liver and/or kidney) or vasospasms. Further examples of disorders which can be treated are renal insufficiency and renal failure, dialysis, subarachnoid hemorrhage, Raynaud's syndrome, portal hypertension and pulmonary hypertension, and the treatment of gastric and duodenal ulcers and of leg ulcer in which vasoconstriction is involved. They can also be employed for atherosclerosis and for preventing restenosis after balloon-induced vasodilatation. Finally, the endothelin concentration is increased in the bronchial secretion of patients with asthma. Increased endothelin levels in blood plasma are also found during migraine attacks. The combination can therefore also be used in these two cases.

Drug therapies, using vascular agents as active ingredients, such as statins, ACE inhibitors, vasodilators, angiotensin II receptor antagonists, diuretics, antithrombolytic agents, beta-adrenergic receptor antagonists, alpha-adrenergic receptor antagonists, calcium channel blockers, and the like, are available for treating cardiovascular and related diseases. The vascular agent can also be administered to treat cardiovascular diseases and other diseases that arise from thrombotic and prothrombotic states in which the coagulation cascade is activated, such as, for example, deep vein thrombosis, disseminated intravascular coagulopathy, Kasabach-Merritt syndrome, pulmonary embolism, myocardial infarction, stroke, thromboembolic complications of surgery, and peripheral arterial occlusion. Methods of the invention may also be useful in the treatment of adult respiratory distress syndrome, septic shock, septicemia, or inflammatory responses, such as edema and acute or chronic atherosclerosis, because thrombin has been shown to activate a large number of cells outside of the coagulation process, such as, for example, neutrophils, fibroblasts, vascular endothelial cells, and smooth muscle cells.

Preferably, the therapeutic vascular agent administered to the patient according to the methods of the subject invention is a statin or ACE inhibitor. Therapeutic vascular agents such as statins and ACE inhibitors are generally used to treat cardiovascular and related diseases, as well as symptoms thereof.

According to the methods of the subject invention, a physician or veterinarian can readily determine a subject who is exhibiting symptoms of any one or more of the diseases described above and can make the determination about which vascular agent is generally suitable for treating specific cardiovascular conditions and symptoms.

For example, myocardial ischemia can be treated by the administration of, for example, a statin, an ACE inhibitor, an angiotensin II receptor antagonist, a calcium channel blocker, an antithrombolytic agent, a beta-adrenergic receptor antagonist, a diuretic, an alpha-adrenergic receptor antagonist, or a mixture thereof. In some instances, congestive heart failure can be treated by the administration of, for example, a statin, an ACE inhibitor, an angiotensin II receptor antagonist, a calcium channel blocker, a vasodilator, a diuretic, or a mixture thereof. Myocardial infarction can be treated by the administration of, for example, a statin, an ACE inhibitor, a calcium channel blocker, an antithrombolytic agent, a beta-adrenergic receptor antagonist, a diuretic, an alpha-adrenergic receptor antagonist, or a mixture thereof. Hypertension can be treated by the administration of, for example, a statin, an ACE inhibitor, a calcium channel blocker, a beta-adrenergic receptor antagonist, a vasodilator, a diuretic, an alpha-adrenergic receptor antagonist, or a mixture thereof. Antithrombolytic agents are used for reducing or removing blood clots from arteries. Hypertrophy can be treated by the administration of, for example, an angiotensin converting enzyme inhibitor, an angiotensin II receptor antagonist, a calcium channel blocker, or a mixture thereof. Ischemia reperfusion injury can be treated by the administration of, for example, an angiotensin converting enzyme inhibitor, an angiotensin II receptor antagonist, a calcium channel blocker, or a mixture thereof. Examples of known angiotensin II receptor antagonists include both angiotensin I receptor subtype antagonists and angiotensin II receptor subtype antagonists. Suitable antiotensin II receptor antagonists include losartan and valsartan.

Suitable calcium channel blockers include, for example, verapamil, diltiazem, nicardipine, nifedipine, amlodipine, felodipine, nimodipine, and bepridil. Antithrombolytic agents known in the art include antiplatelet agents, aspirin, and heparin. Examples of known beta-adrenergic receptor antagonists include atenolol, propranolol, timolol, and metoprolol. Suitable vasodilators include, for example, hydralazine, nitroglycerin, and isosorbide dinitrate. Suitable diuretics include, for example, furosemide, diuril, amiloride, and hydrodiuril. Suitable alpha-adrenergic receptor antagonists include, for example, prazosin, doxazocin, and labetalol. Suitable antioxidants include vitamin E, vitamin C, and isoflavones.

As indicated above, two or more therapeutic vascular agents can be administered concurrently. The terms “concurrent administration” and “concurrently administering” as used herein include administering agents of the invention in admixture, such as, for example, in a pharmaceutical composition or in solution, or as separate compounds, such as, for example, separate pharmaceutical compositions or solutions administered consecutively, simultaneously, or at different times. Optionally, therapeutic vascular agents that are concurrently administered can be selected to act cooperatively, additively, or synergistically with one another. Preferably, if consecutively administered, the consecutive administrations are not so distant in time such that the agents of the invention cannot interact and a lower dosage amount of the active ingredient cannot be administered, if desired.

Selected routes of administration for various therapeutic vascular agents can include: intradermal injection, subcutaneous injection, intramuscular injection, intravenous injection, intraperitoneal injection, intracavitational injection (e.g., injection into a pre-existing physiologic or pathologic body cavity), oral, anal, inhalational, nasal spray, and dermal patch. One skilled in the relevant art can easily select the route most likely to be a therapeutically effective modality for a particular agent.

Regardless of the route of administration selected, the vascular agents administered according to the methods of the present invention can be formulated into pharmaceutically acceptable unit dosage forms by conventional methods known to the pharmaceutical art. An effective but nontoxic quantity of the agent is employed in treatment. The agents can be administered in enteral unit dosage forms, such as, for example, tablets, sustained-release tablets, enteric coated tablets, capsules, sustained-release capsules, enteric coated capsules, pills, powders, granules, solutions, and the like. They can also be administered parenterally, such as, for example, subcutaneously, intramuscularly, intradermally, intramammarally, intravenously, and other administrative methods known in the art.

Although it is possible for a therapeutic vascular agent to be administered alone in a unit dosage form, preferably the agent is administered in admixture as a pharmaceutical composition. A pharmaceutical composition comprises a pharmaceutically acceptable carrier and at least one therapeutic vascular agent. A pharmaceutically acceptable carrier includes, but is not limited to, physiological saline, ringers, phosphate-buffered saline, and other carriers known in the art. Pharmaceutical compositions can also include additives, for example, stabilizers, antioxidants, colorants, excipients, binders, thickeners, dispersing agents, readsorpotion enhancers, buffers, surfactants, preservatives, emulsifiers, isotonizing agents, and diluents. Pharmaceutically acceptable carriers and additives are chosen such that side effects from the pharmaceutical vascular agent are minimized and the performance of the agent is not canceled or inhibited to such an extent that treatment is ineffective.

The ordinarily skilled physician or veterinarian will readily determine and prescribe the therapeutically effective amount of the compound to treat the disease for which treatment is administered. In so proceeding, the physician or veterinarian can employ relatively low dosages at first, subsequently increasing the dose until a maximum response is obtained. Typically, the particular disease, the severity of the disease, the vascular agent to be administered, the route of administration, and the characteristics of the mammal to be treated, for example, age, sex, and weight, are considered in determining the effective amount to administer. Administering a therapeutic amount of a vascular agent for treating cardiovascular and related diseases, or symptoms thereof, is generally in a range of about 0.1-100 mg/kg of a patient's body weight, more preferably in the range of about 0.5-50 mg/kg of a patient's body weight, per daily dose. The agent can be administered for periods of short and long duration. Although some individual situations can warrant to the contrary, short-term administration, for example, 30 days or less, of doses larger than 25 mg/kg of a patient's body weight is preferred to long-term administration. When long-term administration, for example, months or years, is required, the suggested dose should not exceed 25 mg/kg of a patient's body weight.

A therapeutically effective amount of a therapeutic vascular agent for treating the above-identified diseases or symptoms thereof can be administered prior to, concurrently with, or after the onset of the disease or symptom.

### EXAMPLE 1

### Identification of Endothelial Cells

To identify endothelial cells within the blood sample obtained from the patient, an antibody can be utilized. For example, in the case of CECs, the antibody P1H12 can be used. This murine IgG1 monoclonal antibody is obtained by immunizing mice with human umbilical cord endothelial cells (HUVEC), generating a hybridoma line, and separating IgG from supernatants of hybridoma-cell cultures with a protein G column. For some studies fluorescein isothiocyanate-labeled P1H12 prepared with the Fluoro Tag FITC Conjugation kit (SIGMA) can be utilized.

P1H12 reacts specifically with endothelial cells. It stains primary HUVEC and MVEC cultures and the endothelial cells of all vessels in frozen sections of human skin, intestine, ovary, tonsil, lymph node, lung, and kidney. It does not stain any other type of cell in those tissues. It does not stain carcinoma cell lines HT-29 and COL0205, melanoma cell lines A-375 and M21, the T-cell lines Jurkat and HuT78, fibroblasts, HL-60, or Chinese-hamster-ovary cells, or Epstein-Barr virus-transformed B-cell lines. It does not stain monocytes, granulocytes, red cells, platelets, T cells, or B cells from marrow or peripheral blood; nor does it react with marrow megakaryocytes or the megakaryoblast line HU3. The peripheral-blood cells that do stain with P1H12 are also positive for both von Willebrand factor and thrombomodulin (the combined expression of which is limited to endothelium), and they stain for flt and flk (receptors for the endothelial-specific vascular endothelial growth factor). Subgroups of P1H12-positive blood cells also stain for CD34 and two endothelial-specific activation markers (VCAM and E-selectin) (Solovey A. et al., N. Engl J. Med, 1997, 337:1584-1590).

Alternatively, the CD146 antigen (S-Endo 1) can be used to target endothelial cells in blood, as reviewed by Segal, M. S. et al. (Am. J. Physiol. Renal Physiol., July 2002, 283(1):F11-9), the contents of which is incorporated herein by reference in its entirety. CD31, for example, is not a suitable target marker due to expression on platelets and some white blood cells. According to the methods of the subject invention, peripheral blood is collected in a tube containing heparin or EDTA, diluted 1:1 with a solution of phosphate buffered saline (PBS) supplemented with 1 mM EDTA and 0.5% bovine serum albumin and layered on a Ficoll-Hypaque cushion. After a 30-min. spin at 900 g, the red bloods cells and platelets are found in the pellet and the peripheral blood mononuclear cells (PBMC) are in a cushion between the Ficoll-Hypaque and plasma. The PBMC are collected and washed 3 times with PBS/1 mM EDTA/0.5% albumin. B:PBMC can be cytospun onto a slide, fixed, and stained with anti-CD146 monoclonal antibody. The monoclonal antibody is detected by an alkaline phosphatase-conjugated goat anti-mouse antibody and fast red substrate. Alternatively, magnetic beads conjugated with anti-CD146 monoclonal antibody, are added to the PBMC. The CECs can be isolated after lysing red blood cells within whole blood. The PBMC are then precipitated and washed, followed by addition of the magnetic beads to the PBMC. After incubation and washing, only circulating endothelial cells are bound to the magnetic beads. The PBMC can also be cultured on a collagen- or fibronectin-treated tissue culture dish in the presence of vascular endothelial growth factor (VEGF), basic fibroblast growth factor, and hydrocortisone.

Circulating progenitor cells (EPCs) can be isolated using at least one of the following antibodies: anti-CD34, anti-vascular endothelial growth factor receptors-2, and anti-CD133. Preferably, all three antibodies are utilized.

A specific example of magnetic bead that can be utilized to isolate endothelial cells from the blood sample is DYNABEADS. For example, an antibody against CD146 can be coated onto DYNABEADS Pan Mouse IgG (Product number 110.22, 110.23, or 110.24), for example, by simply mixing antibody and beads for 2 hours. Washed beads can be added to the blood sample. 1-2×10⁷ beads per ml blood can be utilized and incubated for 30 minutes at 2-8° C. on a mixer. Bead-bound cells can be separated in a magnetic device (DYNAL MPC) and washed before analysis.

Other methods for isolation of endothelial cells from blood can utilize chip technology, wherein endothelial cell binding ligands, such as antibodies or antibody fragments, are stamped or otherwise immobilized on a matrix and blood is flowed or otherwise contacted with the matrix in vivo or in vitro. The endothelial cells (e.g., molecules on the endothelial cell plasma membrane) interact with the binding ligands on the matrix and are effectively bound to the matrix.

### EXAMPLE 2

### Quantitation of Circulating Endothelial Cells and Endothelial Progenitor Cells

Endothelial cells in the peripheral blood can be enumerated using any of a variety of methods known in the art. For example, endothelial cells in a blood sample can be quantitated by using a flow cytometry procedure, such as that utilized by Mancuso, P. et al. (Blood, 97:3658-3661, 2001) and Monestiroli, S. et al. (Cancer Research, 61:4341-4344, Jun. 1, 2001), the disclosure of which is incorporated herein by reference in its entirety. Briefly, a panel of monoclonal antibodies, including anti-CD45 (to exclude hematopoietic cells), anti-CD31, -CD34, -CD36, -CD105, -CD106, -CD133, and -P1H12 (Solovey, A. et al., N. Engl J. Med, 1997, 337:1584-1590; St. Croix et al., Science, 2000, 289:1197-1202), and appropriate analysis gates are used to enumberate resting and activated endothelial cells. Monoclonal antibodies are conjugated with fluorescein isothiocyanate (FITC), R-phycoerythrin (PE), peridinin chlorophyll protein (PerCP), or allophycocyanin (APC), and cell suspensions are evaluated by a FACSCallibur equipped with a second red-diode laser (BECTON DICKINSON, San Jose, Calif.). Absolute cell numbers are calculated by reference fluorescent beads, and “lyse-no-wash” procedures are used to increase sensitivity and reproducibility. After acquisition of at least 10,000 cells per peripheral blood sample, analysis are considered informative when adequate numbers of events (>100, typically 300-400) are collected in the endothelial cell enumeration gates.

Another method that can be utilized for the quantitation of circulating endothelial cells is immunohistochemical examination, such as that described by Solovey, A. et al. (N. Engl. J. Med., 337:1584-1590, 1997), the disclosure of which is incorporated herein by reference in its entirety.

### EXAMPLE 3

### Determination of Endothelial Cell Activation Phenotype

Vascular disease states which engender the expression of unique disease-associated endothelial membrane components include, for example, hypertension, hypertrophy, arrhythmia, congestive heart failure, myocardial ischemia, heart failure subsequent to myocardial infarction, myocardial infarction, ischemia reperfusion injury, blood coagulation, and platelet aggregation.

Preferably, assessment of the surface phenotype of endothelial cells within a sample is carried out following an enrichment step. Isolation of endothelial cells from blood is described above, as well as in Solevey et al., 1997. Solevey et al. (1997) also describe methods for the detection of antigens that are constitutively expressed by endothelial cells, which may be utilized to measure endothelial cell activation markers according to the methods of the subject invention.

Isolation of plasma membranes from endothelial cells can be accomplished by techniques known in the art for isolation of membranes from cultured vascular endothelial cells, as well. Such techniques include mechanical or hypotonic lysis of cells, followed by equilibrium sedimentation; attachment of nitrocellulose filters to the free surface of adherent cells (preferably previously coated by silica as described in Sambuy, Y. et al., Proc. Nail. Acad. Sci. (USA), 1988, 85:1529-1533), followed by shearing of the plasma membrane attached to the filter; and attachment of magnetic particles to the exterior of the cells (Patton, W. F., Biochem. & Biophys. Acta, 1985, 816:83-92) followed by homogenization and magnetic separation of the resulting plasma-membrane-derived vesicles. One method employs cationic colloidal silica particles (See Chaney et al., J. Biol. Chem., 1983, 258:10062). Briefly, the cell surface is coated with the silica particles, after which sodium polyacrylate is applied as a second coat. The result is to cross-link the silica particles and form a dense pellicle with attached membrane fragments that can be separated from other cellular components by velocity sedimentation. The objective is to abstract from the endothelial cells their plasma membrane components (preferably those on the apical surface of the plasma membrane).

Protein and/or lipid components of the purified membranes are then isolated using well-known methods. The particular technique used for the initial extraction of protein free of lipid, and vice versa, will depend on the subsequent method to be used for resolving different species (protein, lipid, carbohydrate, glycolipid, glycoprotein, etc.), as will be appreciated by those skilled in the art. For example, proteins may be directly solubilized in sodium dodecyl sulfate and urea prior to two-dimensional (2-D) gel electrophoresis and/or column chromatography. Lipids may be extracted in different organic solvents prior to another separation step, e.g., using thin-layer or gas chromatography or column chromatography (e.g., HPLC).

Non-limiting examples of analytical methods for resolving protein species include molecular sieve, ion-exchange, and hydrophobic chromatography; polyacrylamide gel electrophoresis, with or without a prior isoelectric focusing step; and affinity chromatography using materials such as antibodies, or lectins as immobilized specific ligands.

It will be recognized that the methods for comparison of protein and lipid profiles derived from different blood specimens will depend on the analytical methods that produce those profiles. For example, 2-D gel electrophoresis produces complex electropherograms that are best analyzed using commercially available computer imaging software (e.g., MICROSCAN 1000 Technology Resources, Inc., Nashville, Term.). In any case, the result is to identify particular species that are elevated or diminished in the profile derived from the test blood specimen and absent from the control specimen(s).

The methods and kits for the assessment of vascular endothelial function of the subject invention can include either monoclonal or polyclonal antibodies or antibody fragments.

The variable heavy (V_{H}) and variable light (V_{L}) domains of the antibody are involved in antigen recognition. Variable domains of rodent origin can be fused to constant domains of human origin such that the resultant antibody retains the antigenic specificity of the rodent parented antibody (Morrison et al. Proc. Nat. Acad. Sci. USA, 1984, 81:6851-6855). “CDR grafting” can be used to humanize rodent antibodies.

Additionally or alternatively, recombinant monoclonal antibodies may be “primatized,” i.e., antibodies formed in which the variable regions are derived from two different primate species, preferably the variable regions of the antibody from the macaque monkey, and the constant regions from human. The advantages of such antibodies include high homology to human immunoglobulin, presence of human effector functions, reduced immunogenicity and longer serum half-life (Newman et al. Biotechnology, 1992, 10:1455).

Antibody fragments include Fab-like molecules (Better et al. Science, 1988, 240:1041); Fv molecules (Skerra et al. Science, 1988, 240:1038); single-chain Fv (ScFv) molecules where the V_{H} and V_{L} partner domains are linked via a flexible oligopeptide (Bird et al. Science, 1988, 242:423; Huston et al. Proc. Natl. Acad. Sci. USA, 1988, 85:5879) and single domain antibodies (dAbs) comprising isolated V domains (Ward et al. Nature, 1989, 341:544). A general review of the techniques involved in the synthesis of antibody fragments which retain their specific binding sites is found in Winter & Milstein (Nature, 1991, 349:293-299).

Antibody fragments, including but not limited to Fab, (Fab)₂, Fv, scFv and dAb fragments are included in this invention. Antibody-like molecules can be prepared using the recombinant DNA techniques of WO 84/03712. Whole antibodies, and F(ab′)₂ fragments are “bivalent.” By “bivalent” it is meant that the antibodies and F(ab′)₂ fragments have two antigen combining sites. In contrast, Fab, Fv, ScFv and dAb fragments are monovalent, having only one antigen combining site. The art of “antibody engineering” is advancing rapidly, as is described in Tan L. K. and Morrison, S. L. Adv. Drug Deliv. Rev., 1988, 2:129-142, Williams, G. Tibtech, 1988, 6:36-42 and Neuberger, M. S. et al. 8th International Biotechnology Symposium Part 2, 1988, 792-799 (all of which are incorporated herein by reference), and is well suited to preparing antibody-like molecules derived from the antibodies of the invention.

Using standard double antibody detection techniques (e.g., immunoprecipitation and Western blot), CEC activation markers can be identified and quantitated from a blood specimen. Any test which measures the binding of an antigen to an antibody can be used to evaluate the level of antigen or antibody in the patient's blood sample according to the present invention. A number of such tests are known and commonly used commercially.

Immunocytochemistry and immunohistochemistry are techniques that use antibodies to identify antigens on the surface of cells in solution, or on tissue sections, respectively. Immunocytochemistry is used to quantify individual cell populations according to surface markers. Immunohistochemistry is used to localize particular cell populations or antigens. These techniques are also used for the identification of autoantibodies, using tissues or cells that contain the presumed autoantigen as substrate. The antibodies are usually identified using enzyme-conjugated antibodies to the original antibody, followed by a chromagen, which deposits an insoluble colored end product on the cell or tissue.

Another common method of evaluation is a radioimmunoassay, in which radiolabelled reagents are used to detect the antigen or antibody. Antibody can be detected using plates sensitized with antigen. The test antibody is applied and detected by the addition of a radiolabelled ligand specific for that antibody. The amount of ligand bound to the plate is proportional to the amount of test antibody. This test can be reversed to test for antigen. Variations of radioimmunoassays are competition RIA, direct binding RIA, capture RIA, sandwich RIA, and immunoradiometric assay (RMA).

Enzyme linked immunoabsorbent assays (ELISA) are a widely used group of techniques for detecting antigen and antibodies. The principles are analogous to those of radioimmunoassays except that an enzyme is conjugated to the detection system rather than a radioactive molecule. Typical enzymes used are peroxidase, alkaline phosphatase and 2-chractosidase. These can be used to generate colored reaction products from colorless substrates. Color density is proportional to the amount of reactant under investigation. These assays are more convenient than RIA, but less sensitive.

The Western blotting (immunoblotting) method is used to characterize unknown antigens. Components of the biological sample are separated-by gel electrophoresis. SDS gels separate according to molecular weight and IEF gels separate the samples according to charge characteristics. The separated proteins are transferred to membranes (blotted) and identified by immunocytochemistry.

Less often used but suitable methods of evaluation include the Farr assay (in which radiolabelled ligands bind to and detect specific antibody in solution which are precipitated and quantified), precipitin reactions (in which antibodies and antigens crosslink into large lattices to form insoluble immune complexes; only works if antigen and antibody are present in sufficient amounts, at near equivalence, and when there are enough epitopes available to form a lattice); nephelometry (measures immune complexes formed in solution by their ability to scatter light); immunodiffusion (detects antigens and antibodies in agar gels); counter-current electrophoresis (similar to immunodiffusion, except that an electric current is used to drive the antibody and antigen together; useful for low concentrations of antigen or antibody); single radial immunodiffusion (SRID) (quantifies antigens by allowing them to diffuse outward from a well into an antibody containing gel; technique can be reversed by diffusing unknown antibody solutions into an antigen-containing well); rocket electrophoresis (similar to SRID, except that the test antigen is moved into the gel by an electric field); and immunofluorescence (similar to immunochemistry, except that it used fluorescence rather than enzyme conjugates). The antibody used to contact the sample of blood is preferably immobilized onto a solid substrate. The antibody can be immobilized on a solid support or substrate using a variety of means, as described in Antibodies: A Laboratory Manual. Suitable solid substrates include those having a membrane or coating supported by or attached to sticks, synthetic glass, agarose beads, cups, flat packs, or other solid supports. Other solid substrates include cell culture plates, ELISA plates, tubes, and polymeric membranes.

Means for labeling antibodies with detectable agents are also described in Antibodies: A Laboratory Manual, cited supra. The amount of antigen in the patient's blood sample can be determined by any means associated with the selected assay. For example, the selected immunoassay can be carried out with known increasing amounts of antigen to produce a standard curve or color chart, and then the amount of test antigen can be determined by comparing the result of the test to the standard curve or chart that correlates the amount of antigen-antibody complex with known amounts of antigen. The amount of antigen determined to be present in the host biological sample can be used to evaluate the patient's condition in a number of ways. First, the level of antigen can be compared to a population norm based on statistical data. Second, the level of antigen can be considered in light of the patient's own history of antigen level.

Preferred immunoassay techniques use enzyme labels such as horseradish peroxidase, alkaline phosphatase, luciferase, urease, and beta-galactosidase. For example, endothelial cell activation markers conjugated to horseradish peroxidase compete with free sample endothelial activation markers for a limited number of antibody combining sites present on antibodies to endothelial cell activation markers attached to a solid surface, such as a microtiter plate. The endothelial cell activation marker antibodies may be attached to the surface directly, or indirectly, by first coating the surface with multivalent endothelial cell activation marker conjugates (coating antigens) prepared for example by conjugating endothelial cell activation marker with serum proteins such as rabbit serum albumin. After separation of the bound labeled endothelial cell activation marker from the unbound labeled endothelial cell activation marker, the enzyme activity in the bound fraction is determined calorimetrically, for example, by a multi-well microtiter plate reader, at a fixed period of time after the addition of horseradish peroxidase chromogenic substrate.

Alternatively, the antibody, attached to a surface, such as a microtiter plate or polystyrene bead, is incubated with an aliquot of the blood sample. The endothelial activation marker present in the fluid will be bound by the antibody in a manner dependent upon the concentration of endothelial cell activation marker and the association constant between the two. After washing, the antibody/endothelial cell activation marker complex is incubated with a second antibody specific for a different epitope on the endothelial cell activation marker distal enough from the endothelial cell activation marker-specific antibody binding site such that steric hindrance in binding of two antibodies simultaneously to the endothelial cell activation marker may be accomplished. For example, the second antibody may be specific for a portion of a proenzyme sequence, if the endothelial cell activation marker is an enzyme. The second antibody can be labeled in a manner suitable for detection, such as by radioisotope, a fluorescent compound, or a covalently linked enzyme. The amount of labeled secondary antibody bound after washing away unbound secondary antibody is proportional to the amount of endothelial cell activation marker present in the blood sample.

### EXAMPLE 4

### RNA Analysis of Endothelial Cell Activation Markers

Either “fresh” blood or frozen (stored) and subsequently thawed blood may be used for purposes of this invention. Frozen (stored) should optimally be maintained at storage conditions of −20 to −70 degrees centigrade until thawed and used. “Fresh” blood should be refrigerated or maintained on ice until used, with RNA extraction being performed as soon as possible.

Blood can be drawn by standard methods into a collection tube, preferably siliconized glass, either without anticoagulant for preparation of serum, or with EDTA, sodium citrate, heparin, or similar anticoagulants for preparation of plasma. Once the endothelial cells are isolated (e.g., using beads), the intracellular RNA can be extracted for analysis.

Lysis buffer is generally prepared by dissolving 120 grams of guinidine thiocyanate (GuSCN, Fluka Chemical, Buchs, Switzerland) into 100 milliliters of 0.1 M Tris hydrochloride (Tris-HCl) (pH 6.4), and 22 milliliters of 0.2 M EDTA, adjusted to pH 8.0 with NaOH, and 2.6 grams of Triton X-100 (Packard Instrument Co., Downers Grove, Ill. The solution is then homogenized. Washing buffer is prepared by dissolving 120 grams of guinidine thiocyanate (GuSCN) into 100 milliliters of 0.1 M Tris-HCl (pH 6.4).

As an alternative method, intracellular RNA may be extracted from endothelial cells using the Acid Guanidinium Thiocyanate-Phenol-Chloroform extraction method as follows:

The denaturing solution generally consists of 4 M guanidinium thiocyanate, 25 millimolar sodium citrate, pH 7.0, 0.5% sarcosyl, 0.1 M 2-mercaptoethanol. The denaturing solution is prepared as follows: A stock solution is prepared by dissolving 250 grams of guanidinium thiocyanate (GuSCN, Fluka Chemical) with 293 milliliters of demineralized sterile double-distilled water, 17.6 milliliters of 0.75 M sodium citrate, pH 7.0, and 26.4 milliliters of 10% sarcosyl at 65 degrees centigrade. The denaturing solution is prepared by adding 0.36 milliliters 2-mercaptoethanol/50 milliliters of stock solution.

RNA extraction methods previously published for the extraction of mammalian intracellular RNA may be adapted, either as published or with modification, for extraction of endothelial cell marker RNA from endothelial cells. Methods for isolation of RNA and polymerase chain reaction are well known in the art, some of which are described in Sambrook et al. [1989], Molecular Cloning: A Laboratory Manual, 2^{nd} Ed., Cold Spring Harbor Laboratory Press, Cold Spring Harbor, N.Y., the contents of which is incorporated by reference in its entirety.

RNA which has been extracted from endothelial cells, or its corresponding cDNA, can be amplified using any nucleic acid amplification assay utilized for detection of low numbers of RNA molecules. Applicable assays include but are not limited to reverse transcriptase polymerase chain reaction (RT-PCR), ligase chain reaction, branched DNA signal amplification, amplifiable RNA reporters, Q-beta replication, transcription-based amplification, boomerang DNA amplification, strand displacement activation, cycling probe technology, isothermal nucleic acid sequence based amplification (NASBA), and other self-sustained sequence replication assays. It is not necessary to modify these assays from their published methods. The preferred embodiment uses the reverse transcriptase polymerase chain reaction (RT-PCR).

By way of example, following isolation of the endothelial cells from the blood specimen, endothelial cell RNA can be isolated using a cell lysis solution such as that provided by the CELLS-TO-cDNA kit (AMBION, Austin, Tex.). The solution can then be used directly in RT-PCR. Briefly, when using such a kit, a crude cell lysate is subjected to RT-PCR without purifying the RNA. Cells from tissue culture are washed in PBS and then subjected to a heat treatment in a cell lysis buffer. This treatment ruptures the cells, releasing the RNA into the cell lysis buffer, and the heating step inactivates endogenous RNases, protecting the RNA from degradation. Next, the crude cell lysate is treated with DNase I to degrade genomic DNA. After the DNase I is activated by a heating step, the cell lysate is ready for reverse transcription followed by PCR.

### EXAMPLE 5

### Enzymatic Activity of Endothelial Cell Activation Markers

In those instances where the selected endothelial cell activation marker is an enzyme, the enzymatic activity of the marker within the sample can be determined using any of a variety of methods utilized for assaying the activity of the particular enzyme.

For example, in the case of eNOS, enzyme activity can be determined by a modified nitrite assay (Misko, T P et al., Analytical Biochemistry, 214:11-16, 1993; Liao, J K et al., J. Clin. Invest., 96:2661-2666, 1995). Briefly, circulating endothelial cells are treated for 24 h with ox-LDL in the presence and absence of simvastatin (0.1 to 1 mM). After treatment, the medium is removed, and the cells are washed and incubated for 24 h in phenol red-free medium. After 24 h, 300:1 of conditioned medium is mixed with 30:1 of freshly prepared 2,3-diaminonaphthalene (1.5 mmol/L DAN in 1 mol/L HCl). The mixture is protected from light and incubated at 20° C. for 10 min. The reaction is then terminated with 15:1 of 2.8 mol/L NaOH. Fluorescence of 1-(H)-naphthotriazole is measured with excitation and emission wavelengths of 365 and 450 nm, respectively. Standard curves are constructed with known amounts of sodium nitrite. Nonspecific fluorescence is determined in the presence of LNMA (5 mmol/L).

eNOS or iNOS activity can also be measured by incubating endothelial cells with diaminofluorescein (DAF) compounds, such as 4,5-diaminofluorescein-2-diacetate (DAF-2DA) (CALBIOCHEM). DAF compounds are particularly useful indicators for detecting intracellular NO production. DAF-2 is a NO specific indicator that has been widely used for animal cells. DAF-2DA, a derivative of DAF-2, is membrane permeable and can diffuse into the cells. DAF-2DA taken up into the cells can be converted to the activated form (DAF-2) by the activation of intracellular esterases. An increase in fluorescence activity corresponds to NO-dependent conversion of non-fluorescent DAF-2 to green-fluorescent triazolfluorescein (Kojima, H. et al. Anal. Chem., 1998, 70:2446-2453; Fojima, H. et al. Chem. Pharm. Bull., 1998, 46:373-375; Kuo, R. K. et al. Nature, 2000, 406:633-636) Heme oxygenase is another CEC activation marker that is an enzyme. Heme oxygenase catalyzes degradation of heme to iron, biliverdin (a natural antioxidant), and carbon monoxide (CO). Heme oxygenase enzyme activity can be determined, for example, by measurement of iron or CO production, or heme degradation.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for monitoring vascular endothelial function in a patient, comprising:
(a) obtaining a test blood specimen from the patient;
(b) measuring the amount of endothelial cells within the test blood specimen, wherein the endothelial cells are selected from the group consisting of circulating endothelial cells, endothelial progenitor cells, or both circulating endothelial cells and endothelial progenitor cells; and
(c) determining the activation status of the endothelial cells within the test blood specimen.

2. The method of claim 1, further comprising:
(d) correlating the amount of endothelial cells and their activation status with one or more risk factors for disease.

3. The method of claim 1, wherein the risk factors associated with disease are selected from the group consisting of living habits, atherogenic traits, and innate susceptibility.

4. The method of claim 3, wherein the risk factors associated with disease are selected from the group consisting of overeating of saturated fats; high cholesterol; high caloric intake;
physical indolence; cigarette habit; type A behavior; unrestrained weight gain; elevated fibrinogen; leukocytosis; hypertension; genetic predisposition to a disorder selected from the group consisting of dyslipidemia, hypertension, and diabetes; and strong family history of premature cardiovascular disease.

5. The method of claim 1, further comprising:
(d) determining whether the patient is at risk for a disease based upon the activation status of the endothelial cells within the test blood specimen.

6. The method of claim 5, wherein the disease is a vascular disease condition.

7. The method of claim 6, wherein the vascular disease condition is selected from the group consisting of atherosclerosis, hypertension, ischemia, ischemia reperfusion injury, heart failure, arrhythmias, cardiotoxicity, hypertrophy, stroke, deep brain thrombosis, intravascular coagulopathy, Kasabach-Merritt syndrome, pulmonary embolism, myocardial infarction, and thromboembolic complication of surgery.

8. The method of claim 1, wherein the activation status of the endothelial cells is determined by measuring at least one of the endothelial activation markers selected from the group consisting of surface adhesion molecules, procoagulant factors, endothelins, growth factor receptors, nitric oxide, endothelial nitric oxide synthase (eNOS), inducible nitric oxide synthase (iNOS), prostaglandins I2 (PGI2), tissue factors, heme oxygenase (HO), tissue plasminogen activator (tPA), mitochondria superoxide dismutase (MnSOD), Cu/Zn superoxide dismutase (Cu/Zn SOD), tumor growth factor-beta (TGF-β), cyclooxygenase 1 (COX-1), cyclooxygenase-2 (COX-2), vascular cell adhesion molecule (VCAM), vascular endothelial growth factor (VEGF), VEGF receptor, intercellular adhesion molecule (ICAM), E-selectin, and P-selectin.

9. The method of claim 1, wherein the activation status of the endothelial cells is determined by measuring at least one of the endothelial activation markers selected from the group consisting of inducible nitric oxide synthase (iNOS), endothelial nitric oxide synthase (eNOS), vascular cell adhesion molecule (VCAM), and heme oxygenase (HO).

10. The method of claim 1, wherein the endothelial cell activation marker is a vasoactive compound selected from the group consisting of endothelins, nitric oxide, and prostaglandins.

11. The method of claim 1, wherein the endothelial cell activation marker is VCAM-1.

12. The method of claim 1, wherein the endothelial cell activation marker is ICAM-1.

13. The method of claim 1, wherein the endothelial cell activation marker is E-selectin.

14. The method of claim 1, wherein the endothelial cell activation marker is VEGF receptor.

15. The method of claim 1, wherein the amount of endothelial cells within the test blood specimen is measured by flow cytometry.

16. The method of claim 1, wherein at least one of the endothelial cell activation markers is measured by an immunoassay.

17. The method of claim 1, wherein at least one of the endothelial cell activation markers is an enzyme, and wherein the activation status of the endothelial cells is determined by measuring the enzymatic activity exhibited by the enzyme.

18. The method of claim 17, wherein the enzyme is selected from the group consisting of endothelial nitric oxide synthase, inducible nitric oxide synthase, and heme oxygenase.

19. The method of claim 1, wherein at least one of the cardothelial cell activation markers is an enzyme, and wherein the activation status of the endothelial cells is determined by measuring the amount of the enzyme, measuring the amount of RNA encoding the enzyme, and measuring the amount of enzymatic activity exhibited by the enzyme.

20. A method of diagnosing or monitoring a vascular disease in a patient, said method comprising:
(a) administering a therapeutic vascular agent to the patient;
(b) obtaining a test blood specimen from the patient;
(c) measuring the amount of vascular agent within the blood specimen;
(d) measuring the amount of endothelial cells within the test blood specimen, wherein the endothelial cells are selected from the group consisting of circulating endothelial cells, endothelial progenitor cells, and both circulating endothelial cells and endothelial progenitor cells;
(e) determining the activation status of the endothelial cells within the test blood specimen; and
(f) correlating the amount of circulating cells and their activation status with the amount of vascular agent within the blood specimen.

21. The method of claim 20, wherein said method further comprises:
(g) maintaining or modulating the dosage of subsequent administrations of the therapeutic vascular agent to the patient based on the correlation.

22. The method of claim 20, wherein the activation status of the circulating endothelial cells is determined by measuring at least one of the circulating endothelial cell activation markers selected from the group consisting of surface adhesion molecules, procoagulant factors, endothelins, growth factor receptors, nitric oxide, endothelial nitric oxide synthase (eNOS), inducible nitric oxide synthase (iNOS), prostaglandins 12 (PGI2), tissue factors, heme oxygenase (HO), tissue plasminogen activator (tPA), mitochondria superoxide dismutase (MnSOD), Cu/Zn superoxide dismutase (Cu/Zn SOD), tumor growth factor-beta (TGF-β), cyclooxygenase 1 (COX-1), cyclooxygenase-2 (COX-2), vascular cell adhesion molecule (VCAM), intercellular adhesion molecule (ICAM), vascular endothelial growth factor (VEGF), VEGF receptor, E-selectin, and P-selectin.

23. The method of claim 20, wherein the activation status of the endothelial cells is determined by measuring at least one of the endothelial cell activation markers selected from the group consisting of inducible nitric oxide synthase (iNOS), endothelial nitric oxide synthase (eNOS), vascular cell adhesion molecule (VCAM), and heme oxygenase (HO).

24. The method of claim 20, wherein the endothelial cell activation marker is a vasoactive compound selected from the group consisting of endothelins, nitric oxide, and prostaglandins.

25. The method of claim 20, wherein the endothelial cell activation marker is VCAM-1.

26. The method of claim 20, wherein the endothelial cell activation marker is ICAM-1.

27. The method of claim 20, wherein the endothelial cell activation marker is E-selectin.

28. The method of claim 20, wherein the therapeutic vascular agent is selected from the group consisting of angiotensin converting enzyme inhibitors, angiotensin II receptor antagonists, diuretics, alpha-adrenoreceptor antagonists, cardiac glycosides, phosphodiesterase inhibitors, beta-adrenoreceptor antagonists, calcium channel blockers, 3-hydroxy-3-methylglutaryl-coenzyme A reductase inhibitors, imidazoline receptor blockers, endothelin receptor blockers, and organic nitrites.

29. The method of claim 20, wherein the therapeutic vascular agent is an angiotensin converting enzyme inhibitor.

30. The method of claim 20, wherein the angiotensin converting enzyme inhibitor is selected from the group consisting of captopril, zofenopril, enalapril, lisinopril, quinapril, ramapril, perindopril, cilazapril, benazapril, fosinopril, trandolopril, benazeprilat, delapril, fentiapril, imidapril, libenzapril, moexipril, pentopril, perindopril, pivopril, quinaprilat, spirapril, spiraprilat, ceronapril, enalapril, indolapril, and alacepril.

31. The method of claim 20, wherein the therapeutic vascular agent is a 3-hydroxy-3-methylglutaryl-coenzyme A reductase inhibitor.

32. The method of claim 20, wherein the 3-hydroxy-3-methylglutaryl-coenzyme A reductase inhibitor is selected from the group consisting of lovastatin, simvastatin, pravastatin, fluvastatin, atorvastatin, and cerivastatin.

33. The method of claim 20, wherein the patient has or is suspected of having a vascular disease.

34. The method of claim 33, wherein the vascular disease is selected from the group consisting of hypertension, hypertrophy, arrhythmia, congestive heart failure, myocardial ischemia, myocardial infarction, heart failure subsequent to myocardial infarction, ischemia reperfusion injury, blood coagulation, and platelet aggregation.

35. The method of claim 20, wherein the amount of therapeutic vascular agent within the test blood specimen is measured by an immunoassay.

36. The method of claim 20, wherein the amount of circulating endothelial cells within the test blood specimen is measured by flow cytometry.

37. The method of claim 22, wherein at least one of the endothelial cell activation markers is measured by an immunoassay.

38. The method of claim 22, wherein at least one of the endothelial cell activation markers measured is an enzyme, and wherein the activation status of the endothelial cells is determined by measuring the enzymatic activity exhibited by the enzyme.

39. The method of claim 38, wherein the enzyme is endothelial nitric oxide synthase.

40. The method of claim 22, wherein at least one of the circulating endothelial cell activation markers is an enzyme, and wherein the activation status of the circulating endothelial cells is determined by measuring the amount of the enzyme, measuring the amount of RNA encoding the enzyme, and measuring the amount of the enzymatic activity exhibited by the enzyme.

41. A method of diagnosing or monitoring a vascular disease in a patient, said method comprising:
(a) administering a therapeutic vascular agent to the patient;
(b) obtaining a test blood specimen from the patient;
(c) measuring the amount of vascular agent within the blood specimen;
(d) measuring the amount of RNA encoding an endothelial cell activation marker within the test blood specimen;
(e) measuring the amount of endothelial cell activation marker enzymatic activity within the test blood specimen, if the endothelial cell activation marker is an enzyme;
(f) measuring the amount of endothelial cells within the test blood specimen, wherein the endothelial cells are selected from the group consisting of circulating endothelial cells, endothelial progenitor cells, and both circulating endothelial cells and endothelial progenitor cells; and
(g) correlating at least one of the following with the amount of endothelial cells and the amount of therapeutic vascular agent within the test blood specimen: (i) amount of RNA encoding an endothelial cell activation marker; and (ii) amount of endothelial cell activation marker enzymatic activity.

42. The method according to claim 41, further comprising maintaining or modulating the dosage of subsequent administrations of the therapeutic vascular agent to the patient based upon the correlation.

43. The method according to claim 42, further comprising comparing the amounts of endothelial cells and therapeutic vascular agent and at least one of the following within the test blood specimen with that of a control blood specimen: (i) amount of RNA encoding a endothelial cell activation marker; and (ii) amount of endothelial cell activation marker enzymatic activity.

44. The method according to claim 41, wherein a higher amount of RNA encoding an endothelial cell activation marker per endothelial cell or higher amount of endothelial cell activation marker enzymatic activity per unit of endothelial cells indicates the presence of vascular disease.

45. A kit for monitoring vascular endothelial function comprising at least two of the components selected from the group consisting of:
(a) means for measuring the amount of endothelial cells within a test blood specimen, wherein said endothelial cells are selected from the group consisting of circulating endothelial cells, endothelial progenitor cells, and both circulating endothelial cells and endothelial progenitor cells.
(b) means for measuring the amount of a therapeutic vascular agent within the test blood specimen;
(c) means for measuring the amount of RNA encoding an endothelial cell activation marker within the test blood specimen; and
(d) means for measuring the amount of endothelial cell activation marker enzymatic activity within the test blood specimen.

46. The kit of claim 45, wherein said means for measuring the amount of endothelial cells comprises at least one antibody or antibody fragment specific for an antigen selected from the group consisting of P1H12, CD146, CD34, VEGF receptor-2, and CD133.

47. The kit of claim 45, wherein said means for measuring the amount of RNA comprises a primer.

48. The kit of claim 45, wherein said means for measuring the amount of said therapeutic vascular agent comprises an antibody or antibody fragment that recognizes said therapeutic vascular agent.

49. The kit of claim 45, wherein said means for measuring the amount of endothelial cell activation marker enzymatic activity comprises an enzymatic substrate of the endothelial cell activation marker.
